(19) United States
(12) Patent Application Publication
(10) Pub. No.: US 2022/0211623 A1
(43) Pub. Date: Jul. 7, 2022

(54) INHALABLE SUSTAINED RELEASE COMPOSITION OF BRONCHODILATOR FOR USE IN TREATING PULMONARY DISEASE

(71) Applicant: InspirMed Corp., Taipei City 115 (TW)

(72) Inventors: Keelung HONG, South San Francisco, CA (US); Jonathan FANG, South San Francisco, CA (US); Yun-Long TSENG, Taipei City (TW); Wan-Ni YU, Taipei City (TW); Ting-Yu CHENG, Taipei City (TW); Jo-Hsin TANG, Taipei City (TW)

(21) Appl. No.: 17/610,346

(22) PCT Filed: May 14, 2020
(86) PCT No.: PCT/US2020/032799

§ 371 Date: Nov. 10, 2021

Related U.S. Application Data
(60) Provisional application No. 62/847,613, filed on May 14, 2019.

(51) Int. Cl. A61K 9/127; A61K 47/28; A61K 47/24; A61K 31/5386; A61K 31/439; A61K 31/4704; A61K 31/137; A61K 31/40; A61K 9/00; A61P 11/06

(52) U.S. Cl. CPC A61K 9/1271; A61K 47/28; A61K 47/24; A61K 31/5386; A61P 11/06; A61K 31/4704; A61K 31/137; A61K 31/40; A61K 9/0073; A61K 31/439

(57) ABSTRACT

Provided is a liposomal sustained release composition of bronchodilator for use in treatment of pulmonary disease. The liposomal bronchodilator has a liposome containing a bronchodilator entrapped in the liposome. The bronchodilator has been stably encapsulated in the liposome, and the resulting liposomal bronchodilator is proven to be stably aerosolized or nebulized for administration via the inhalation route to treat a subject in need thereof.

### RELATED APPLICATIONS

This application claims the benefit of U.S. Provisional Application No. 62/847,613, filed May 14, 2019, the contents of which are incorporated by reference in their entirety.

### BACKGROUND

### Technical Field

The present invention relates to an inhalable drug delivery system for delivery of a sustained-release liposomal composition. The present invention relates to a method of preparing the drug delivery system. The present invention also relates to a sustained-release pharmaceutical composition adapted to pulmonary delivery system, which has a prolonged duration of efficacy.

### Description of Related Art

Undesirable pulmonary diseases are initiated from various external effectors and become overwhelming issues for the aging society. Chronic obstructive pulmonary disease (COPD) is an extremely serious, debilitating lung disease that leads to early death among all. COPD is characterized by inflammation and thickening of mucus in airways, degradation of air sacs, and deteriorating lung function. Common symptoms of COPD include chronic cough, dyspnea, and tightness in the chest.

There are four different classes of potential drugs for treating pulmonary diseases, particularly to COPD: corticosteroids, bronchodilators, specifically anticholinergic agents (specifically muscarinic antagonists) and β₂ adrenergic receptor agonists, antibiotics and mucolytics. Currently, inhalation therapy is the preferred administration route for COPD treatment. The Global Initiative for Chronic Obstructive Lung Disease (GOLD) recommends bronchodilators as first-line, standard drug therapy for most COPD patients. In addition, GOLD recommends nebulized inhalation therapy for specific patient populations, such as the elderly and patients with low inspiratory flow rates.

Two long-acting muscarinic antagonist (LAMA) that have been and are currently being used for treating COPD are tiotropium bromide and glycopyrrolate. Tiotropium bromide is available as a dry powder with a recommended dose of 18 μg twice-daily (Spiriva® HandiHaler®, NDA No. 21395, Boehringer Ingelheim) or as an inhalation solution at a dose of 2.5 μg twice-daily (Spiriva® Respimat®, NDA No. 207070, Boehringer Ingelheim). Glycopyrrolate is available as a dry powder with a recommended dose of 15.6 μg twice-daily (Seebri® Neohaler®, NDA No. 207923, Novartis). In addition, a nebulized glycopyrrolate inhalation solution was recently approved by the FDA with a recommended dose of 25 μg twice-daily (Lonhala® Magnair®, NDA No. 208437, Sunovion).

In addition, there are various drug combinations that are seeing extensive use for treating COPD. One such dual COPD drug product combines glycopyrrolate and indacaterol, an ultra-long-acting β₂ adrenergic receptor agonist (ultra-LABA), with recommended doses of 15.6 μg and 27.5 μg, respectively, twice-daily (Utibron® Neohaler®, NDA No. 207930, Novartis). Another COPD drug combination contains tiotropium bromide and olodaterol, another ultra-LABA, with recommended doses of 3 μg and 2.7 μg, respectively, twice-daily (Stiolto® Respimat®, NDA No. 206756, Boehringer Ingelheim). Side effects of COPD treatment with these drugs include tremors, tachycardia, dizziness, allergic reactions, blurry vision, and throat irritation.

Liposomes have been utilized as drug carriers for sustained drug delivery for decades. Liposome encapsulation of drug substance alters the pharmacokinetic profile of the free drug substance, provides slow drug release systemically or at a local physical environment, allows for high administered doses with less frequent drug administration, and possibly reduces side effects and toxicity. High drug substance encapsulation inside liposome can be achieved via a remote loading method (also known as active loading), which relies on transmembrane pH and ion gradients to allow for diffusion of free, uncharged drug substance into the liposome. While inside the liposome, the free drug substance can form complex with a trapping agent (a counterion in the aqueous interior) to even precipitate into a drug-counterion salt that stays inside the liposome. A liposomal drug formulation can be tailored to achieve slow drug release in vivo, which would prolong the therapeutic effect of the drug. This can be accomplished by adjusting the liposome formulation and optimizing certain liposome properties, such as the phospholipids used (different chain lengths, phase transition temperatures), lipid to cholesterol ratio, amount of polyethylene glycol (PEG) on the liposome (to evade clearance by macrophage), trapping agent used for drug substance encapsulation, and possibly the lamellarity of the liposome.

Whilst a drug has been stably encapsulated in the liposome, the resulting liposomal drug formulation is in question to be definitely able to be aerosolized or nebulized for inhalation delivery. It is not readily apparent that utilizing liposome technology to reformulate bronchodilators can yield a liposomal drug formulation for inhalation at a therapeutic dose to treat COPD and other related pulmonary diseases.

Currently, there have been no practicable liposomal drug formulations for inhalation as drug products for treating COPD and other related diseases. Regarding current liposomal drug formulations for inhalation therapy, there are two inhalable liposomal drug products in development that have reached clinical trials: liposomal amikacin (Insmed, Inc.) and liposomal ciprofloxacin (Aradigm Corporation). Both liposomal antibiotics for inhalation are being investigated for treating multiple respiratory diseases, such as cystic fibrosis (CF), non-CF bronchiectasis, nontuberculous mycobacterial lung disease, and other virulent infections. Recently, Arikayce® (amikacin liposome inhalation suspension) received accelerated approval by the FDA for the treatment of Mycobacterium avium complex lung disease. Both liposomal drug formulations for inhalation therapy are designed for antibiotics to easily access microorganism or infected tissues by modifying lipid content to be electrically neutral (See U.S. Pat. No. 8,226,975) or by adjusting particle size and amount of free ciprofloxacin to attenuate attraction of macrophage (See U.S. Pat. No. 8,071,127).

Unfortunately, the existing inhalable liposomal compositions are unable to anticipate the unmet needs for treatment of other pulmonary diseases, such as COPD, which may necessitate a drug product with different target product profiles, such as deep lung deposition, enhanced mucus penetration, prolonged drug retention in the lung, increased liposomal drug stability, and so forth. To date, the relevant study of effective inhalable drug for treatment of COPD or the like in a form of lipid based sustained release composition has not been reported yet. Therefore, there remains an unmet need for a sustained release formulation with a predetermined encapsulation efficiency to achieve a balance by reducing dosing frequency for bronchodilator such as anticholinergic agents and/or β₂ adrenergic receptor agonists and targeting a desired therapeutic window. In addition, the formulation suitable for COPD and other related pulmonary diseases should have the following properties: being inhalable, having an improved stability or resistance to destruction by local lung surfactant, and furthermore, having desired dose strength to ensure the potential for reaching the desired efficacy in the pulmonary environment. The present invention addresses this need and other needs.

### SUMMARY

The present invention provides an inhalable liposomal drug formulation comprising phospholipid(s), optionally a sterol and/or PEG-modified phospholipid, and a bronchodilator, particularly to an anticholinergic agent, more particularly to a quaternary ammonium muscarinic antagonist such as tiotropium bromide, encapsulated in the aqueous interior of the liposome.

To improve upon existing treatment paradigms of pulmonary disease, such as COPD, and take advantage of the benefits of slow, sustained drug release, we developed a sustained release composition of bronchodilator comprising liposomal bronchodilator and a predetermined amount of free bronchodilator in an aqueous suspension that can be aerosolized and inhaled for enhanced treatment of pulmonary disease. Particularly, there is a need for inhalable sustained release formulations for COPD treatment.

The present invention provides the liposomal bronchodilator for use in treatment of pulmonary disease, particularly to COPD, having the advantages of: 1) achieving a longer therapeutic effect compared to inhaled free drug substance, 2) delivering the drug directly to the disease site, 3) quicker onset of action, 4) reducing adverse drug reactions and systemic effects, 5) bypassing first-pass metabolism observed in oral dosing, thus increasing the bioavailability of the drug substance (and possibly reducing hepatotoxicity), 6) increasing the residence time of the drug substance in lung via sustained release from liposomal drug, 7) decreasing the frequency of drug administration, 8) non-invasive inhalation delivery, and 9) improving patient outcomes and compliance.

The bronchodilator according to the present invention is encapsulated in the liposome by remote loading using a trapping agent composed of an ammonium compound and an anionic counterion to achieve the sustained release composition with a preferred release profile and reduced toxicity.

The liposomal bronchodilator according to the present invention optionally incorporates a significant amount of PEG moiety onto the surface of the vesicles to achieve longer, sustained drug release that will be safe, efficacious, and suitable for once-daily or even less frequent dosing.

In a particular embodiment, the liposomal bronchodilator comprises phosphocholine (PC):cholesterol at a molar ratio of 1:1 to 3:2, wherein the PC can be hydrogenated soy phosphatidylcholine (HSPC), 1,2-distearoyl-sn-glycero-3-phosphocholine (DSPC), 1,2-dipalmitoyl-sn-glycero-3-phosphocholine (DPPC), or a mixture thereof, such as DSPC and 1,2-dipalmitoyl-sn-glycero-3-phosphoethanolamine (DPPE) at a molar ratio of 1:1.

In a particular embodiment, the PEG-modified phosphoethanolamine (PE) can be DSPE-PEG2000 and ranges from 0.0001 mol % to 40 mol % of the total lipid content of the liposomes.

In some embodiments, the lipid concentration of the liposomal bronchodilator of the sustained release composition ranges from 10 to 25 mM and the drug-to-lipid (D/L) ratio ranges from 0.01 mol/mol to 5 mol/mol.

In some embodiments, the mean particle diameter of the liposomal bronchodilator ranges from 50 nm to 1,000 nm.

In a particular embodiment, the liposomal bronchodilator comprises an anticholinergic agent or a β2 adrenergic receptor agonist.

In some embodiments, the liposomal bronchodilator comprises the bronchodilator selected from the group consisting of tiotropium bromide, glycopyrrolate, umeclidinium bromide, aclidinium bromide, ipratropium bromide, oxitropium bromide, revefenacin, and indacaterol, arformoterol, formoterol, olodaterol, salbutamol, salmeterol, vilanterol and combinations thereof.

In some embodiments, the liposomal bronchodilator comprises a quaternary ammonium muscarinic antagonist.

In another aspect, the present invention also provides an aerosolized composition of particles of liposomal bronchodilator for use in treatment of pulmonary disease, which has a drug-to-lipid ratio of at least 0.1 mol/mol.

In another aspect, the present invention also provides an aerosolized composition of particles containing the liposomal bronchodilator for use in treatment of pulmonary disease, which comprises the sustained release composition for use according to the present invention.

Other objectives, advantages and novel features of the invention will become more apparent from the following detailed description when taken in conjunction with the accompanying drawings.

### BRIEF DESCRIPTION OF THE DRAWINGS

FIG. 1 is a graph depicting the in vitro release profiles of the liposomal tiotropium sustained release formulations with various compositions.

FIG. 2 is a line graph showing the changes in body weight in 3 groups of LPS-treated mice subjected to either instillation of liposomal tiotropium with various trapping agents or no treatment.

FIG. 3 is a line graph showing the survival rates of 3 groups of LPS-treated mice subjected to either instillation of liposomal tiotropium with various trapping agents or no treatment.

### DETAILED DESCRIPTION OF THE PREFERRED EMBODIMENTS

As employed above and throughout the disclosure, the following terms, unless otherwise indicated, shall be understood to have the following meanings.

As used herein, the singular forms “a”, “an” and “the” include the plural reference unless the context clearly indicates otherwise.

All numbers herein may be understood as modified by “about,” which, when referring to a measurable value such as an amount, a temporal duration, and the like, is meant to encompass variations of ±10%, preferably ±5%, more preferably ±1%, and even more preferably ±0.1% from the specified value, as such variations are appropriate to obtain a desired amount of liposomal drug, unless other specified.

The term “treating” “treated” or “treatment” as used herein includes preventive (e.g. prophylactic), palliative, and curative uses or results. The term “subject” includes a vertebrate having cancer or other diseases. Preferably, the subject is a warm-blooded animal, including mammals, preferably humans.

As used herein, the term drug to lipid ratio refers to the ratio of bronchodilator to total phospholipid content. The bronchodilator content of free and liposomal drug was determined by UV-Vis absorbance measurements. The phospholipid content, or concentration, of liposome and liposomal drug was determined by assaying the phosphorus content of liposome and liposomal drug samples using a phosphorus assay (adapted from G. Rouser et al., Lipids 1970, 5, 494-496).

### Pulmonary Diseases

Pulmonary diseases in accordance with the present invention include, but are not limited to: chronic obstructive pulmonary disease (COPD), COPD-related diseases, such as chronic bronchitis and emphysema, asthma, exercise induced bronchospasm, cystic fibrosis and atelectasis. Symptoms typically include chronic cough, dyspnea, and tightness in the chest, and gradual onset of shortness of breath. Complications include pulmonary hypertension, heart failure, pneumonia, or pulmonary embolism.

### Liposome

The term “liposome” or “liposomal” as used herein are directed to a particle characterized by having an aqueous interior space sequestered from an outer medium by a membrane of one or more bilayer membranes forming a vesicle. Bilayer membranes of liposomes are typically formed by one or more lipids, i.e., amphiphilic molecules of synthetic or natural origin that comprise spatially separated hydrophobic and hydrophilic domains. In certain embodiments of the present invention, the term “liposomes” refers to small unilamellar vesicle (SUV) in which one lipid bilayer forms the membrane.

In general, liposomes comprise a lipid mixture typically including one or more lipids selected from the group consisting of: dialiphatic chain lipids, such as phospholipids, diglycerides, dialiphatic glycolipids, single lipids such as sphingomyelin and glycosphingolipid, sterols such as cholesterol and derivates thereof, and combinations thereof.

Examples of phospholipids according to the present invention include, but are not limited to, 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1-palmitoyl-2-stearoyl-sn-glycero-3-phosphocholine (PSPC), 1-palmitoyl-2-oleoyl-sn-glycero-3-phosphatidylcholine (POPC), 1,2-distearoyl-sn-glycero-3-phosphocholine (DSPC), 1,2-dioleoyl-sn-glycero-3-phosphocholine (DOPC), hydrogenated soy phosphatidylcholine (HSPC), 1,2-dimyristoyl-sn-glycero-3-phospho-(1′-rac-glycerol) (sodium salt) (DMPG), 1,2-dipalmitoyl-sn-glycero-3-phospho-(1′-rac-glycerol) (sodium salt) (DPPG), 1-palmitoyl-2-stearoyl-sn-glycero-3-phospho-(1′-rac-glycerol) (sodium salt) (PSPG), 1,2-distearoyl-sn-glycero-3-phospho-(1′-rac-glycerol) (sodium salt) (DSPG), 1,2-dioleoyl-sn-glycero-3-phospho-(1′-rac-glycerol) (DOPG), 1,2-dimyristoyl-sn-glycero-3-phospho-L-serine (sodium salt) (DMPS), 1,2-dipalmitoyl-sn-glycero-3-phospho-L-serine (sodium salt) (DPPS), 1,2-distearoyl-sn-glycero-3-phospho-L-serine (sodium salt) (DSPS), 1,2-dioleoyl-sn-glycero-3-phospho-L-serine (DOPS), 1,2-dimyristoyl-sn-glycero-3-phosphate (sodium salt) (DMPA), 1,2-dipalmitoyl-sn-glycero-3-phosphate (sodium salt) (DPPA), 1,2-distearoyl-sn-glycero-3-phosphate (sodium salt) (DSPA), 1,2-dioleoyl-sn-glycero-3-phosphate (sodium salt) (DOPA), 1,2-dipalmitoyl-sn-glycero-3-phosphoethanolamine (DPPE), 1-palmitoyl-2-oleoyl-sn-glycero-3-phosphoethanolamine (POPE), 1,2-distearoyl-sn-glycero-3-phosphoethanolamine (DSPE), 1,2-dioleoyl-sn-glycero-3-phosphoethanolamine (DOPE), 1,2-dipalmitoyl-sn-glycero-3-phospho-(1′-myo-inositol) (ammonium salt) (DPPI), 1,2-distearoyl-sn-glycero-3-phosphoinositol (ammonium salt) (DSPI), 1,2-dioleoyl-sn-glycero-3-phospho-(1′-myo-inositol) (ammonium salt) (DOPI), cardiolipin, L-α-phosphatidylcholine (EPC), and L-α-phosphatidylethanolamine (EPE).

### Polyethylene Glycol (PEG)-Modified Lipid

The polyethylene glycol-modified lipid comprises a polyethylene glycol moiety conjugated with a lipid. In some embodiments, the PEG moiety has a molecular weight from about 1,000 to about 20,000 daltons. In a particular embodiment, the PEG-modified lipid is mixed with the phospholipids to form liposomes with one or more bilayer membranes. In some embodiments, the amount of PEG-modified lipid ranges from 0.0001 mol % to 40 mol %, optionally from 0.001 mol % to 30 mol %, optionally from 0.01 mol % to 20 mol %; and particularly no more than 6 mol %, optionally 5 mol %, 3 mol % or 2 mol %, on the basis of the total phospholipids and sterol. In some embodiments, the PEG-modified lipid has a PEG moiety with an average molecular weight ranging from 1,000 g/mol to 5,000 g/mol. In a particular embodiment, the PEG-modified lipid is phosphatidylethanolamine linked to a polyethylene glycol group (PE-PEG). In further embodiments, PEG-modified phosphatidylethanolamine is 1,2-distearoyl-sn-glycero-3-phosphoethanolamine-N-[methoxy(polyethylene glycol)] (DSPE-PEG).

### Liposomal Bronchodilators

The terms “liposomal bronchodilator” and “liposomal drug” are interchangeably used in the present disclosure. The liposomal bronchodilator in accordance to the present invention comprises liposomes with entrapped bronchodilator, which are prepared by encapsulating the bronchodilator in the aqueous interior of the liposome via a transmembrane pH gradient-driven remote loading method. In some embodiments, the transmembrane pH gradient is created by using a trapping agent for remote loading of the bronchodilator into liposome and the trapping agent is composed of an ammonium compound and an anionic counterion.

The term “ammonium compound” includes non-substituted or substituted ammonium being a cationic ion presented by NW′, wherein each R is independently H or an organic residue, and the organic residue is independently alkyl, alkylidene, heterocyclic alkyl, cycloalkyl, aryl, alkenyl, cycloalkenyl, or a hydroxyl-substituted derivative thereof, optionally including within its hydrocarbon chain a S, O, or N atom, forming an ether, ester, thioether, amine, or amide bond. In one embodiment, the ammonium compound is ammonium.

The term “anionic counterion” refers to an anionic ion or an entity which is covalently linked to one anionic functional group. The anionic ion or the anionic functional group has negative electric charge under physiological environment.

The anionic ion or the anionic functional group can be selected from one or more of the following: sulfate, citrate, sulfonate, phosphate, pyrophosphate, tartrate, succinate, maleate, borate, carboxylate, bicarbonate, glucoronate, chloride, hydroxide, nitrate, cyanate or bromide.

In one embodiment, the anionic ion and the anionic functional group is selected from one or more of the following: citrate, sulfate, sulfonate, phosphate, pyrophosphate, or carboxylate.

In yet another embodiment, the entity linked to the anionic functional group can be a natural or synthetic, organic or inorganic compound. Examples of the entity include, but are not limited to, a non-polymer substance selected from alkyl group or aryl group, such as benzene, nucleotide and saccharide. The alkyl refers to a saturated hydrocarbon radical having indicated number of carbon atoms. For example, the alkyl is selected from the group consisting of alkyl of 1 to 4 carbons (C₁₋₄ alkyl), alkyl of 1 to 6 carbons (C₁₋₆ alkyl), alkyl of 1 to 8 carbons (C₁₋₈ alkyl), alkyl of 1 to 10 carbons (C₁₋₁₀ alkyl), alkyl of 1 to 12 carbons (C₁₋₁₂ alkyl), alkyl of 1 to 14 carbons (C₁₋₁₄ alkyl), alkyl of 1 to 16 carbons (C₁₋₁₆ alkyl), alkyl of 1 to 18 carbons (C₁₋₁₈ alkyl) and alkyl of 1 to 20 carbons (C₁₋₂₀ alkyl).

In some embodiments, the anionic counterion is selected from the group consisting of sulfate, phosphate, citrate and combinations thereof.

In some embodiments, the trapping agent is selected from the group consisting of ammonium sulfate, ammonium phosphate, ammonium citrate, dimethylammonium sulfate, dimethylammonium phosphate, dimethylammonium citrate, diethylammonium sulfate, diethylammonium phosphate, diethylammonium citrate, trimethylammonium sulfate, trimethylammonium phosphate, trimethylammonium citrate, triethylammonium sulfate, triethylammonium phosphate, triethylammonium citrate and combinations thereof.

In some embodiments, the sustained release composition according to the present invention, wherein the liposomal bronchodilator has a mean particle diameter between 50 nm and 1,000 nm. Non-limiting examples of liposomes has an average diameter ranges from 50 nm to 20 μm, 50 nm to 10 μm, 50 nm to 1000 nm, 50 nm to 500 nm, 50 nm to 400 nm, 50 nm to 300 nm, 50 nm to 250 nm, or 50 nm to 200 nm.

The term “bronchodilator” refers to a substance dilating the bronchi or bronchioles, thus allowing for increased airflow to the lungs.

In some embodiments, the bronchodilator is directed to anticholinergic agents and β₂ adrenergic receptor agonists. Among bronchodilators, quaternary ammonium muscarinic antagonists including tiotropium bromide, glycopyrrolate, umeclidinium bromide, aclidinium bromide, ipratropium bromide, and oxitropium bromide are commonly used anticholinergic agents which bind to muscarinic receptor(s) on the airway smooth muscle and block cholinergic contractile action. Since quaternary ammonium muscarinic antagonist are fully ionized, they are poorly absorbed into the bloodstream and do not cross the blood-brain barrier, resulting in limiting the anticholinergic effect at the site of delivery without causing systemic adverse effects.

In some embodiments, the bronchodilator, in accordance with the present invention is a β₂ adrenergic receptor agonist selected from the group consisting of indacaterol, arformoterol, formoterol, olodaterol, salbutamol, salmeterol, and vilanterol.

In one aspect, the liposomal bronchodilator comprises:

a lipid bilayer comprising: one or more phospholipids, a sterol, and an optional polyethylene glycol (PEG)-modified lipid, particularly to PEG-modified phosphatidylethanolamine (PEG-PE); and
an aqueous interior encompassed by the lipid bilayer and containing one or more bronchodilators.

In an embodiment, the one or more phospholipids is neutral phospholipid, and the polyethylene glycol (PEG)-modified lipid is DSPE-PEG. The amount of DSPE-PEG ranges from 0.001 to 5 mol % on the basis of the total phospholipid and sterol.

### Aerosolized Particles of the Sustained Release Composition

The sustained release composition in accordance with the present invention is adapted to preparation of an aerosolized composition of particles. In one embodiment, the liposomal bronchodilator comprises: a lipid bilayer comprising: a phospholipid, a sterol, and a PEG-modified phosphatidylethanolamine; and an aqueous interior encompassed by the lipid bilayer and containing the bronchodilator, and wherein drug leakage of the liposomal bronchodilator from the liposome after aerosolization is less than 10%.

In one embodiment, the sustained release composition of bronchodilator for use according to the present invention has a lipid concentration ranging from 1 to 25 mM. In one embodiment, the sustained release composition has a concentration of the bronchodilator ranging from 0.1 mg/mL to 30 mg/mL, 0.5 mg/mL to 20 mg/mL, 1 to 15 mg/mL and 2 mg/mL to 10 mg/mL. In one embodiment, the sustained release composition of bronchodilator for use according to the present invention has a drug-to-phospholipid ratio at least 0.1 mol/mol, and preferably ranging from 0.05 mol/mol to 1 mol/mol, optionally 0.1 mol/mol to 0.7 mol/mol, optionally 0.15 mol/mol to 0.6 mol/mol and optionally 0.15 mol/mol to 0.2 mol/mol. In some embodiments, free bronchodilator of the sustained release composition is at an amount less than 50%, 45%, 40%, 35%, 30%, 25%, 20%, 15%, 10% or 5% of the total amount of the bronchodilator of the sustained release composition.

In some embodiments, aerosolized composition of particles according to the sustained release composition of the present disclosure is generated by a nebulizer, which is selected from the group consisting of air-jet nebulizer, ultrasonic nebulizer and a vibrating mesh nebulizer.

In some embodiments, the aerosolized composition of particles has a mass median aerodynamic diameter between 0.5 μm and 5 μm, and optionally 1 μm and 3 μM.

In a specific embodiment, the aerosolized composition of particles is subjected to pulmonary delivery to a subject in need to perform a release rate between about 0.5% and 25% of the administered drug dose per hour with complete release of the bronchodilator occurring after a minimum of about 12 to 24 hours.

The disclosure will be further described with reference to the following specific, non-limiting examples.

### EXAMPLES

The following examples illustrate the preparation and properties of certain embodiments of the present invention.

### Example 1 Stability of the Liposomal Bronchodilator

### A. Preparation of Liposomal Tiotropium

### I. Preparation of Empty Liposomes

Liposomes were prepared via the thin-film hydration method or solvent injection method. The process for preparing empty liposomes by thin-film hydration method is embodied by the method comprising the following steps:

1. weighing out lipid mixture of phospholipids, cholesterol at a predetermined molar ratio in the presence of DSPE-PEG2000 and adding them to 10 mL of chloroform in a round-bottom flask;
2. placing the flask in a rotary evaporator at 60° C. and stirring the flask to dissolve the lipid mixture, followed by putting the flask under vacuum while stirring to evaporate off the chloroform to obtain a dried lipid film;
3. preparing a trapping agent solution (e.g. ammonium sulfate (A.S.)) by adding a trapping agent to distilled water and vortexing the solution to dissolve the powder;
4. adding the trapping agent solution to the dried lipid film and stirring it at 60° C. for 30 minutes to form a proliposome solution;
5. freeze-thawing the proliposome solution for 5 times with liquid nitrogen and 60° C. water bath to obtain a liposome sample;
6. extruding obtained liposome sample 10 times through 0.2 μm polycarbonate membrane, then 10 times through 0.1 μm polycarbonate membrane at 60° C.;
7. dialyzing the extruded liposome sample to remove free trapping agent, followed by adding the sample to a dialysis bag (MWCO: 25 kD), sealing the bag, and stirring the dialysis bag in 100× volume of a 9.4% (w/v) sucrose solution; and further replacing the sucrose solution after 1 hour, 4 hours, and let it stir overnight; and
8. sterilizing the dialyzed liposome sample by filtering it through a 0.45 μm PTFE membrane to obtain the empty liposomes.
II. Drug Loading of Tiotropium into Liposome to Obtain Liposomal Tiotropium

The following method represents a typical protocol for the encapsulation of tiotropium in liposome by remote loading, which comprises steps of:

1. preparing solutions of 9.4% (w/v) sucrose and 9.4% (w/v) sucrose containing 200 mM L-histidine (L-His) buffer, pH 7.68, for maintaining the pH under loading condition;
2. preparing a solution of 10 mg/mL of tiotropium bromide in 9.4% (w/v) sucrose and briefly heating it at 60° C. to obtain a stock solution containing tiotropium bromide (hereafter denoted as Tio stock solution);
3. mixing together empty liposomes as prepared by the process according to Example 1, section (I) [in a typical embodiment, with the condition of: HSPC:cholesterol:DSPE-PEG2000 at a molar ratio of 3:2:0.045, 300 mM ammonium sulfate (A.S.), and 20 mM lipid concentration], a 9.4% (w/v) sucrose solution, a 9.4% (w/v) sucrose buffer, pH 7.68, ethanol (final of 10% by volume in mixture), and Tio stock solution into a conical tube to obtain a loading solution, targeting a D/L ratio to be 100 g/mol;
4. continuously shaking the loading solution at 60° C. for 60 minutes to form the liposomal drug sample, followed by placing the liposomal drug sample on ice for a few minutes;
5. removing the ethanol and free drug by diluting the loading solution 10-fold in a 9.4% sucrose solution containing 40 mM L-His, loading the diluted sample into an Amicon Ultra-15 centrifugal filter unit (MWCO: 100 kD) and centrifuging the sample at 3,800 g for 80 minutes, then finally adjusting the concentration of tiotropium bromide to 1 mg/mL with a 9.4% sucrose solution containing 40 mM L-His; and
6. determining the drug encapsulation (i.e. loading efficiency) of the final sample using size-exclusion column chromatography and HPLC analysis (drug concentrations of all samples, liposomal or total form, were determined by absorbance measurements at the wavelength 237 nm).

### B. The Effect of Different Trapping Agents on Drug Loading Profile

Different trapping agents were used to encapsulate the bronchodilators including both anticholinergic agents and β₂ adrenergic receptor agonists. The preparations of liposomal drug formulations were performed according to the above Section A (same lipid bilayer composition of HSPC:cholesterol:DSPE-PEG2000 at a molar ratio of 3:2:0.045), except for using the following trapping agents: (1) 75 mM of triethylammonium sucrose octasulfate, and (2) 300 mM of ammonium sulfate. The results from the drug loading experiments are shown in Table 1.

TABLE 1
The drug loading profile of different trapping agents

| | | | Encapsulation | Average |
|---|---|---|---|---|
| | Trapping | Purified D/L | Efficiency | Particle |
| Bronchodilator | Agent | (mole/mole) | (%) | Size (nm) |
| Tiotropium | 1 | 0.16 | 76.1 | 118 |
| bromide | | | | |
| Tiotropium | 1 | 0.19 | 88.5 | 141 |
| bromide | | | | |
| Tiotropium | 2 | 0.19 | 91.6 | 110 |
| bromide | | | | |
| Glycopyrrolate | 1 | 0.3 | 54 | 200 |
| Indacaterol | 1 | 0.67 | 80 | n.d. |
| maleate | | | | |
| Indacaterol | 2 | 0.57 | 87.3 | n.d. |
| maleate | | | | |
| Salbutamol | 1 | 0.46 | 64.1 | 201 |
| hemisulfate | | | | |
| Salbutamol | 2 | 0.49 | 73.2 | 203 |
| hemisulfate | | | | |

n.d., not determined.

### C. Storage Stability of Liposomal Drug

The stability of liposomal tiotropium stored at 4° C. was monitored for at least two months. Tiotropium was loaded into empty liposomes with 75 mM of triethylammonium sucrose octasulfate (TEA-SOS) as trapping agent 1 to obtain the liposomal drug sample. After storage of the liposomal drug sample at 4° C. for over two months, there was no drug leakage out of the liposome. Regarding the mean particle diameter, the liposomal drug composed of 3:2:0.045 HSPC:cholesterol:DSPE-PEG2000 molar ratio (0.9 mol % PEG) remained approximately 140 nm over time. In terms of encapsulation stability, the D/L ratio of the liposomal drug remained approximately 0.19 (mol/mol) over time. Regarding the encapsulation stability of liposomal drug composed of 3:2 HSPC:cholesterol, the D/L ratio remained approximately 0.14 (mol/mol)).

### Example 2 Releasing Profile of the Liposomal Bronchodilator

### A. Preparation of Liposomal Tiotropium

### I. Preparation of Empty Liposomes

Liposomes were prepared via the thin-film hydration method or solvent injection method. The process for preparing empty liposomes by solvent injection method is embodied by the method comprising the following steps:

1. weighing out lipid mixture of phospholipids, cholesterol and the DSPE-PEG2000 at a predetermined molar ratio (the details of compositions shown in table 2) and adding them to ethanol in a glass tube and dissolving the lipid mixture at 60° C.;
2. pre-warming the indicated trapping agent solution ((1) 75 mM of triethylammonium sucrose octasulfate, (2) 300 mM of ammonium sulfate) at 60° C. for at least 30 minutes;
3. adding the dissolved lipid mixture by syringe into the pre-warmed trapping agent solution under stirring to form pro-liposome sample and then keep stirring the pro-liposome sample at 60° C. for 5 minutes;
4. extruding the pro-liposome sample through a 0.2 μm polycarbonate membrane at 60° C., and then through a 0.1 μm polycarbonate membrane at 60° C. to obtain liposome sample;
5. dialyzing the extruded liposome sample to remove external medium from liposomes of the liposome sample using a dialysis bag (MWCO: 25 kDa) and 9.4% (w/v) of sucrose as the dialysis solution with the volume of 100 times of liposome sample; replacing the sucrose solution two times between 4-hour and 8-hour-stirring intervals; and
6. sterilizing the dialyzed liposome sample by filtering it through a 0.2 μm polytetrafluoroethylene (PTFE) membrane to obtain the empty liposomes.
II. Drug Loading of Tiotropium into Liposome to Obtain Liposomal Tiotropium

The following method represents a typical protocol for the encapsulation of tiotropium in liposome by remote loading, which comprises steps of:

1. preparing solutions of 9.4% (w/v) sucrose and 9.4% (w/v) sucrose buffer containing 200 mM L-histidine (L-His), pH 7.68;
2. preparing a solution of 10 mg/mL of tiotropium bromide in 9.4% (w/v) sucrose;
3. mixing together empty liposomes as prepared by the process (in a typical embodiment, with the condition of: HSPC:cholesterol at a molar ratio of 3:2, 300 mM ammonium sulfate (A.S.), and 20 mM lipid concentration), a 9.4% (w/v) sucrose solution, a 9.4% (w/v) sucrose buffer, pH 7.68, with a final concentration of 40 mM L-His, ethanol (final of 10% by volume in mixture), and Tio stock solution into a conical tube to obtain a loading solution, targeting a D/L ratio to be 100 g/mol; and
4. continuously shaking the loading solution at 60° C. for 60 minutes to form the liposomal drug sample, followed by placing the liposomal drug sample on ice for a few minutes.

### III. Optional Post-Insertion DSPE-mPEG on Liposomal Tiotropium

1. preparing 11.25 mM stock solution of DSPE-mPEG in 9.4% (w/v) sucrose,
2. mixing DSPE-mPEG stock solution with the liposomal tiotropium (from section II) at predetermined concentration of total lipid at 3%; and
3. continuously shaking the liposome solution with DSPE-mPEG at 60° C. for 5 minutes, followed by placing the liposomal drug sample on ice for a few minutes.

### IV. Removal of Free Form and Ethanol

1. diluting the liposomal drug sample (with or without DSPE-mPEG) 10-fold by 9.4% sucrose with 40 mM L-histidine buffer in a centrifuge tube (Amicon Ultra-15 centrifugal filter unit (MWCO: 100 kD)) and centrifuging the sample at 3,800 g for 80 minutes for removing the ethanol and free drug, then finally adjusting the concentration of tiotropium bromide to 1 mg/mL with a 9.4% sucrose solution, containing 40 mM L-His; and
2. determining the drug encapsulation (i.e. loading efficiency) of the final sample using UV-Vis plate reader (drug concentrations of all samples, liposomal or total form, were determined by absorbance measurements at the wavelength 237 nm). The results are shown below in Table 2.

TABLE 2
The drug loading profile of different trapping agents

| | | | DSPE- | Purified | Encapsulation | Average |
|---|---|---|---|---|---|---|
| Formulation | Phospholipids:cho- | Trapping | mPEG | D/L | Efficiency | Particle |
| No. | lesterol:DSPE-mPEG | Agent | (%) | (mole/mole) | (%) | Size (nm) |
| A | 3:2:0.045 | 1 | 0.9 | 0.20 | 91.2 | 141 |
| B | 3:2:0.15 | 1 | 3 | 0.22 | 85.9 | 141 |
| C | 3:2:0 | 2 | 0 | 0.20 | 93.2 | 118 |
| D | 3:2:0.15 | 2 | 3 | 0.13 | 79.3 | 118 |

### B. In Vitro Drug Release in Simulated Lung Fluid

The release profile experiments of liposomal tiotropium with or without PEG content in simulated lung fluid (SLF) were performed to demonstrate their sustained release properties. The test articles (the prepared samples of liposomal tiotropium) were prepared at about the same amount of tiotropium bromide (1 mg/mL drug) with only a small amount of free drug present in each sample (0.01˜10% of the total drug content). The protocol for the in vitro release (IVR) experiments is outlined as follows:

1. diluting the test article 10-fold by mixing 0.5 mL of each sample of the liposomal tiotropium with 4.5 mL of SLF (pre-warmed at 37° C.) and placing the diluted sample in a 15-mL centrifuge tube;
2. placing the centrifuge tubes containing the diluted samples onto sample wells of a Intelli-mixer rotator and rotating at 20 rpm, incubating at 37° C.; and
3. sampling 1 mL of the diluted samples at predetermined time points for analyzing encapsulated efficiency (0, 4, and 24 hours).

The analytical method for determining the tiotropium encapsulation efficiency is as follows:
a. packing and washing 2 mL of a G50 column with a 9.4% sucrose solution (less than 5 mL);
b. adding 0.2 mL of the sample to the column, then adding 0.15 mL of a 9.4% sucrose solution three separate times and waiting for the solution to be eluted out;
c. adding 0.7 mL of 9.4% sucrose solution to the column and collecting the eluent (as liposomal form) in a 1.5 mL Eppendorf; then transferring and mixing 0.24 mL of the eluent with 0.96 mL methanol;
d. in a separate 1.5 mL Eppendorf tube placing 0.2 mL of the unpurified sample and adding 0.5 mL 9.4% sucrose and mixing well, then transferring 0.24 mL of the solution and mixing with 0.96 mL methanol (as total form);
e. centrifuging the pre and post-column samples (the liposome form and the total form) at 20,600 g for 10 minutes; and
f. measuring the absorbance of the final, supernatant of the samples at the wavelength 237 nm using a UV-Vis plate reader to determine the drug concentrations of each sample.

The encapsulation efficiency (EE) of the bronchodilator in the liposomes was calculated and obtained by the formula: the liposomal form (LF) of the drug divided by the total form (TF) of the drug:

EE (%)=LF/TF*100%.

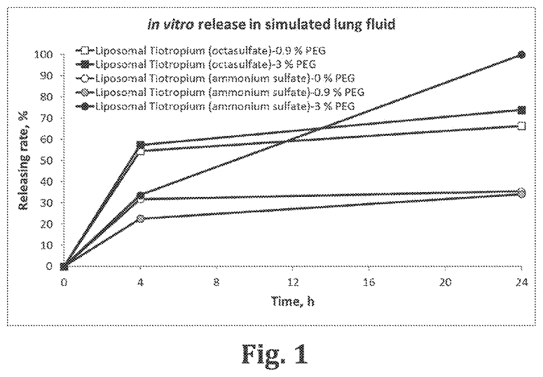

The releasing profile was plotted as FIG. 1, depicting the releasing rate (%) versus time. The releasing rate was calculated by the formula: the initial liposomal form minus liposomal form at each time point and then divided by the initial liposomal form:

(LFₜ₀−LFₜ)/LFₜ₀*100%.

The liposomal tiotropium with trapping agents 1 or 2 exhibited slow releasing profiles in SLF. The liposomal tiotropium with trapping agent 1 and 0.9% and 3% PEG, exhibited stable and slow releasing profiles and up to 50% of the initial drug content in SLF was released over 24 hours. On the other hand, the liposomal tiotropium with trapping agent 2, in the absence or presence of a small amount (0.9%) of PEG, exhibited a slower releasing profile (slow drug release and only up to 30% of the initial drug content in SLF was released over 24 hours), compared to the liposomal tiotropium composed of 3% PEG-DSPE. A prolonged release profile of drug substance is desired for improved efficacy and treatment of with lower dosing frequency. Therefore, we chose the two liposomal tiotropium formulations with slower releasing profiles among all formulations and used them in the following toxicity study. The two selected formulations, A and C from Table 2, had low and zero content of DSPE-mPEG, respectively, and were derived from two different trapping agents.

### Example 3 Toxicity Evaluation of Inhaled Liposomal Tiotropium in an LPS-Induced Acute COPD Animal Model

The toxicity of two liposomal tiotropium formulations in lipopolysaccharide (LPS)-induced acute COPD mice was investigated. The study design is given in Table 3. Briefly, twelve mice were divided into three groups (N=4). The untreated control group is only symptom-mimicking and mice from control group did not receive any treatment.

The description of each composition is given below:

Group #1: Liposomal tiotropium formulation from the Example 2: Liposomes loaded with tiotropium with 300 mM ammonium sulfate (AS) as trapping agent. The formulation comprises a lipid concentration of 20 mM, tiotropium concentration of 1 mg/mL;
Group #2: Liposomal tiotropium formulation from the Example 2: Liposomes loaded with tiotropium with 75 mM TEA-SOS as trapping agent. The formulation comprises a lipid concentration of 20 mM, tiotropium concentration of 1 mg/mL; Group #3: Untreated control group, only LPS induction.

The acute COPD animal model was established by IT instillation of LPS at 1 mg/kg on day 0. After 24 hours, twenty-five μL of liposomal tiotropium with different trapping agents (Group #1 and Group #2, N=4) were instilled intratracheally to the LPS-treated mice for the toxicity evaluation, in comparison to the untreated control (Group #3, N=4). The dosing regimens are shown in Table 3. Both formulations were administered once at a dose of 1 mg/kg. Body weight and survival were recorded during the study period.

TABLE 3
Study design of liposomal tiotropium toxicity
in the acute COPD animal model

| Group | | | Dose | |
|---|---|---|---|---|
| # | Composition | Animal no. | frequency | Dosage |
| 1 | AS-Tio 1.06 mg/mL | 4 | Single | 1 mg/kg |
| | | | injection | |
| 2 | TEA-Tio 1.07 | 4 | Single | 1 mg/kg |
| | mg/mL | | injection | |
| 3 | Untreated control | 4 | N/A | N/A |

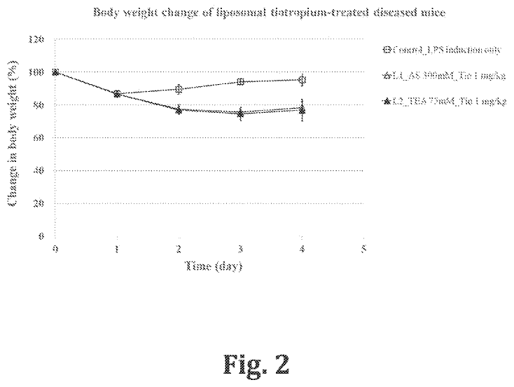

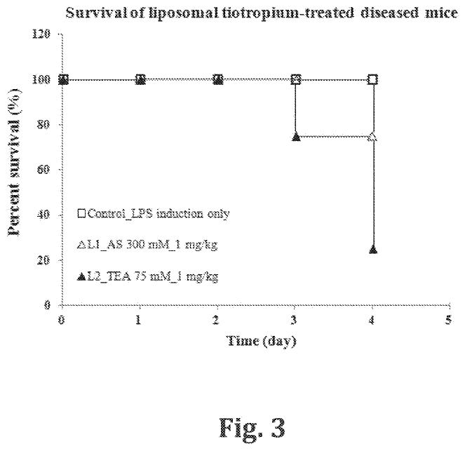

Toxicity was determined by body weight and survival of the animals at the endpoint. Referring to FIG. 2, over twenty percent body weight loss was observed in both Group #1 and Group #2, comparing with Group #3. The survival rate of Group #2 (25%) was less than that of Group #1 (75%), suggesting a milder toxicity and a more tolerable outcome when using ammonium sulfate as the trapping agent, as illustrated in FIG. 3. As a result, although equal total amounts of tiotropium were administrated by intratracheal instillation, the liposomal tiotropium group with ammonium sulfate in the formulation showed reduced side effects compared to the TEA-SOS formulation group in the treatment of COPD.

1. A sustained release composition of bronchodilator comprising a liposomal bronchodilator, wherein the liposomal bronchodilator comprises:
a lipid bilayer comprising: one or more phospholipids and a sterol; and
an aqueous interior encompassed by the lipid bilayer and containing a bronchodilator encapsulated in the liposome by remote loading using a trapping agent, wherein the trapping agent is composed of an ammonium compound and an anionic counterion, and the anionic counterion is an anionic ion or an entity which is covalently linked to one anionic functional group.

2. A method of treating pulmonary disease in a subject in need thereof, comprising administering to the subject an effective amount of the sustained release composition of bronchodilator for use of claim 1, wherein the pulmonary disease includes chronic obstructive pulmonary disease (COPD) or COPD-related diseases, symptoms, or complications.

3. The sustained release composition of bronchodilator of claim 1, wherein the molar ratio of the total phospholipids to sterol ranges from 1:1 to 3:2.

4. The sustained release composition of bronchodilator of claim 3, wherein the sterol is cholesterol.

5. The sustained release composition of bronchodilator of claim 3, wherein the one or more phospholipids are selected from the group consisting of hydrogenated soy phosphatidylcholine (HSPC), 1,2-distearoyl-sn-glycero-3-phosphocholine (DSPC), 1,2-dipalmitoyl-sn-glycero-3-phosphocholine (DPPC), phosphatidylethanolamine lipid, such as 1,2-dipalmitoyl-sn-glycero-3-phosphoethanolamine (DPPE), and combinations thereof.

6. The sustained release composition of bronchodilator of claim 1, wherein the lipid bilayer comprises a polyethylene glycol (PEG)-modified lipid at an amount ranging from 0.0001 mol % to 40 mol %, optionally less than 6 mol %, optionally ranging from 0.001 mol % to 30 mol % on the basis of the total phospholipids and sterol.

7. The sustained release composition of bronchodilator of claim 6, wherein the PEG-modified lipid has a PEG moiety with an average molecular weight ranging from 1,000 g/mol to 5,000 g/mol.

8. The sustained release composition of bronchodilator of claim 6, wherein the PEG-modified lipid is 1,2-distearoyl-sn-glycero-3-phosphoethanolamine-N-[methoxy(polyethylene glycol)] (DSPE-PEG).

9. The sustained release composition of bronchodilator of claim 8, wherein the one or more phospholipids are neutral phospholipids, the amount of DSPE-PEG of the liposome ranges from 0.001 to 5 mol % on the basis of the total phospholipid and sterol.

10. The sustained release composition of bronchodilator of claim 1, wherein the liposomal bronchodilator has a mean particle diameter between 50 nm and 1,000 nm.

11. The sustained release composition of bronchodilator of claim 1, wherein the bronchodilator is an anticholinergic agent or a β₂ adrenergic receptor agonist.

12. The sustained release composition of bronchodilator of claim 1, wherein the bronchodilator is selected from the group consisting of tiotropium bromide, glycopyrrolate, umeclidinium bromide, aclidinium bromide, ipratropium bromide, oxitropium bromide, revefenacin, and indacaterol, arformoterol, formoterol, olodaterol, salbutamol, salmeterol, vilanterol, and combinations thereof.

13. The sustained release composition of bronchodilator of claim 1, wherein the bronchodilator is a quaternary ammonium muscarinic antagonist.

14. The sustained release composition of bronchodilator of claim 13, wherein the quaternary ammonium muscarinic antagonist is selected from the group consisting of tiotropium bromide, glycopyrrolate, umeclidinium bromide, aclidinium bromide, ipratropium bromide, and oxitropium bromide.

15. The sustained release composition of bronchodilator of claim 1, which has a lipid concentration ranging from 1 to 25 mM.

16. The sustained release composition of bronchodilator of claim 1, which has a concentration of the bronchodilator ranging from 1 to 15 mg/mL.

17. The sustained release composition of bronchodilator of claim 1, which has a drug-to-phospholipid ratio ranging from 0.01 mol/mol to 1 mol/mol, optionally 0.1 mol/mol to 0.7 mol/mol, optionally 0.15 mol/mol to 0.6 mol/mol, and optionally 0.15 mol/mol to 0.2 mol/mol.

18. A sustained release composition of bronchodilator comprising a liposomal bronchodilator, wherein the liposomal bronchodilator comprises:
a lipid bilayer comprising: one or more phospholipids and cholesterol; and
an aqueous interior encompassed by the lipid bilayer and containing tiotropium encapsulated in the liposome by remote loading using a trapping agent, wherein the trapping agent is composed of an ammonium compound and an anionic counterion, and the anionic counterion is selected from the group consisting of sulfate, citrate, sulfonate, phosphate, pyrophosphate, tartrate, succinate, maleate, borate, carboxylate, bicarbonate, glucoronate, chloride, hydroxide, nitrate, cyanate, bromide, and combinations thereof.

19. An aerosolized composition of particles comprising multiple particles of the sustained release composition of bronchodilator according to claim 1.

20. The aerosolized composition of particles of claim 19, wherein the multiple particles have a mass median aerodynamic diameter ranging from about 0.5 μm to 5 μm.
